(19) United States
(12) Patent Application Publication
(10) Pub. No.: US 2006/0099581 A1
(43) Pub. Date: May 11, 2006

(54) Method for analysis of methylated nucleic acids

(76) Inventor: Kurt Berlin, Stahnsdorf (DE)

Correspondence Address: LAWRENCE HARBIN;MCINTYRE HARBIN & KING LLP, 500 9TH STREET, S.E., WASHINGTON, DC, 20003, US

(21) Appl. No.: 10/510,698

(22) PCT Filed: Apr. 9, 2003
(86) PCT No.: PCT/IB03/01791

§ 371 Date: Oct. 8, 2004

Related U.S. Application Data
(60) Provisional application No. 60/370,690, filed on Apr. 9, 2002.

(51) Int. Cl. C12Q 1/68; C12P 19/34; C07H 21/04

(52) U.S. Cl. 435/6; 435/91.2; 536/25.32

(57) ABSTRACT

A method is disclosed providing analysis of the degree of methylation within nucleic acid samples, including the degree of methylation within CpG islands. After bisulfite treatment of a nucleic acid sample to convert cytosines to uracils, multiple species of paired oliogonucleotide primers and optionally a methylation insensitive reference primer pair are used to amplify target sequences within the sample by methylation specific PCR. Amplification of multiple primer pairs is combined with the use of a real time PCR. Amplificates of primer pairs are detected and quantified by comparison, thus allowing for a detailed, more specific, and quantifiable analysis of the methylation status within a complex CpG methylation pattern of a nucleic acid sample. Primer pairs and a kit are also disclosed.

### BACKGROUND

The present invention relates to analysis of methylated nucleic acids, and more particularly to analysis of the methylation status of a nucleic acid sample within a complex methylation pattern.

Pathogenic states are known to be expressed by a modified methylation pattern of individual genes or of the genome. 5-methylcytosine is the most frequent covalently modified base in the DNA of eukaryotic cells, and plays a role in the regulation of transcription, in genetic imprinting, and in tumorigenesis. Aberrant methylation patterns have been shown to be implicated in a wide variety of disease states. The identification of 5-methylcytosine sites in a specific specimen is thus of considerable interest, not only for research, but particularly for the molecular diagnosis of various diseases. The ability to characterize a tissue type, either diseased or healthy, based on its methylation pattern requires that techniques for the analysis of complexes of CpG positions be developed.

Furthermore, although a healthy tissue may often be distinguished from a cancerous tissue by means of co-methylation analysis, a more sophisticated method of tumor methylation analysis would be useful. For example, tumor tissue samples often consist of both tumor and adjacent tissue. Where classification of the tumor tissue type is required to be carried out, the analysis of the methylation state of multiple CpG sites will have to be carried out for both methylated and unmethylated variants of each CpG position. However, anaylsis using conventional MSP techniques would be time conuming and impractical. Another use for the analysis of multiple CpG site methylation state analysis is the observation of disease progression. If a disease condition is identified by the aberrant methylation state of CpG rich islands, then the analysis must address CpG positions within a tissue or cell sample that may contain both methylated and unmethylated copies of the same CpG position. Investigation of such a sample using techniques such as bisulphite sequencing and MSP analysis is again time consuming and impractical.

The covalent attachment of a methyl group at the C⁵-position of the nucleotide base cytosine is particularly common within CpG dinucleotides of gene regulatory regions. The frequency of occurrence of any particular dinucleotide in a given DNA sequence is 1/16 or ˜6%. However, in humans the average genomic measured frequency of the CpG dinucleotide is very low—about 1/70. Nonetheless, contiguous genomic regions of between 300 bp and 3000 bp in length exist in which the occurrence of CpG dinucleotides is significantly higher than normal. These CpG-rich regions are referred to in the art as CpG “islands” and represent about 1% of the genome. Such CpG islands have primarily been observed in the 5-region of genes, and more than 60% of human promoters are contained in, or overlap with, such CpG islands.

The most frequently applied method for investigating DNA or other nucleic acid samples for the presence of 5-methylcytosine is based on the specific reaction of bisulfite with cytosine. The bisulfite reaction selectively converts cytosine—but not 5-methylcytosine—to uracil, which corresponds in its base-pairing behavior to thymidine. After DNA treatment with bisulfite reaction, 5-methylcytosine can be detected by standard molecular biological techniques as the single remaining cytosine—for example, by amplification and hybridization or sequencing—whereas 5-methylcytosine cannot be distinguished in an untreated DNA sample from cytosine by means of its hybridization behavior.

A treated DNA sample can be analyzed using polymerase chain reaction (PCR) based methods, including PCR combined with bilsufite treatment of DNA to convert unmethylated cytosines to uracil. However, the reduced sequence complexity of bisulphite treated DNA makes nucleic acid analysis more difficult using standard molecular biological techniques such as PCR and hybridization analysis. Typical problems with PCR include increased levels of cross-hybridization, mis-priming, false positive results for methylation, and difficulty in identifying hypermethylated alleles in small samples.

In overcoming disadvantages of the use of PCR and bisulfite treatment for detection of methylated nucleic acids, Herman et al. (U.S. Pat. No. 5,786,146) described the use of methylation sensitive primers and a method for the detection of a methylated CpG within a nucleic acid—methylation specific PCR (“MSP”). Herman et al. describe the use of oligonucleotide primer pairs specific for methylated versus unmethylated alleles in nucleic acids for the amplification of DNA samples, the presence or absence of an amplificate thereby indicating the methylation status of a group of CpG positions within a CpG island of the sample—i.e., whether any one CpG position within a group of positions covered by the forward and reverse primers is methylated

The method is disclosed by Herman et al. as suited only to the detection of an unmethylated versus a methylated position within CpG-rich regions. The method is less amenable to the analysis of complex methylation patterns or to the quantification of the degree of methylation at a specific CpG position within a sample. The method is described as allowing only a semi-quantitative assessment of the degree of methylation, and is therefore not suited to a detailed analysis of hypermethylated DNA within promoter regions. The use of MSP has proved particularly useful in the detection of hypermethylated regulatory regions of genes. However, the method as disclosed by Herman et al. is suited only to the detection of hypermethylated versus non-methylated positions within CpG rich regions. This method is less amenable to the analysis of complex methylation patterns) or the quantification of the degree of methylation at a specific CpG position within a sample.

In particular, current methods of CpG methylation analysis, including bisulphite treatment followed by nucleic acid amplification using methylation specific PCR (MSP) and methylation specific primers, have not shown the use of multiple species of primers in order to quantify the degree of methylation within a sample. As disclosed in Laird et al. (U.S. Pat. No. 6,331,393 B1), the method of Herman et al. is a qualitative, not quantitative, technique. The use in MSP of two separate two different PCR reactions (methylated and unmethylated) presents difficulties in making kinetic comparisons between the reactions. Furthermore, the use in MSP of paired primers, which often cover a DNA sequence containing more than one CpG dinucleotide, may represent only one of multiple possible sequence variants. Therefore, the method of Herman et al. is described as non-quantitative, since it is based on the occurrence or non-occurrence of a PCR product in the fully methylated versus fully unmethylated reaction.

It can therefore be seen that for the analysis of complex methylation patterns, MSP is not a versatile technique. For the investigation of the methylation status of a sequence that may comprise both methylated and non-methylated cytosine positions, a large number of primers covering every possible combination of methylated and non-methylated CpG positions has to be designed and tested. This is economically impractical, time consuming, expensive, and labor intensive. These problems would become more apparent were the method to be applied in a high throughput setting, where the maintenance of data quality may be difficult to control and false annealing of primers may lead to incorrect data interpretation. A further drawback of the method as described by Herman et al. is that although the methods enables the detection of methylated sequences within a sample, it does not allow quantification or absolute measurement of the amount of methylated sequences present in a sample.

Therefore, an improved method of CpG methylation status assessment is required for, among other reasons, providing a reliable, quantitative means to allow the analysis of the degree of methylation at a specific CpG position and to enable the simultaneous analysis of the methylation status of all CpG dinucleotides within a sample, thereby advancing the investigation of the complex epigenomic significance of multiple CpG sites upon phenotypic variation.

### SUMMARY OF THE INVENTION

The method according to the invention provides a means for the analysis of complex methylation patterns within biological samples by use of multiple pairs of methylation specific primers. After unmethylated cytosine bases in a nucleic acid sample are converted into uracil bases is provided by a converting agent that does not change methylated cytosines, selected segments of the converted nucleic acid sample are amplified in a polymerase reaction wherein at least two oligonucleotide primer pairs are employed, such that the amplificates formed are differentially detectable and quantifiable. One primer pair binds preferentially to treated nucleic acid that was initially methylated in the sequence the primer is hybridizing to. Another primer pair binds preferentially to treated nucleic acid that was initially unmethylated in the sequence the primer is hybridizing to. A third oligonucleotide primer pair may be used to amplify a sequence that acts as a reference sequence. The degree of methylation in at least one selected segment of the nucleic acid sample is determined based on comparative differences in amplificates formed from each of the oligonucleotide primer pairs. The invention also includes primers and a kit.

### BRIEF DESCRIPTION OF DRAWINGS

FIG. 1 is a diagram showing a method of MSP amplification according to the prior art.

FIG. 2 is a flow chart of a preferred embodiment of the invention.

### DESCRIPTION OF ILLUSTRATIVE EMBODIMENTS

An embodiment of the invention provides a method for analysis of methylation patterns within nucleic acids, including analysis of CpG rich islands. Multiple species of primer oligonucleotides are used to amplify a nucleic acid sample. The comparative amounts of amplificates are detected, thus allowing for a detailed, more specific, and quantifiable analysis of the methylation status of a nucleic acid sample within a complex methylation pattern. According to the invention, a complex methylation pattern is taken to mean the degree of methylation at one or more CpG positions, wherein the degree of methylation is determined by the relative amount of methylated to non-methylated nucleic acids in the sample.

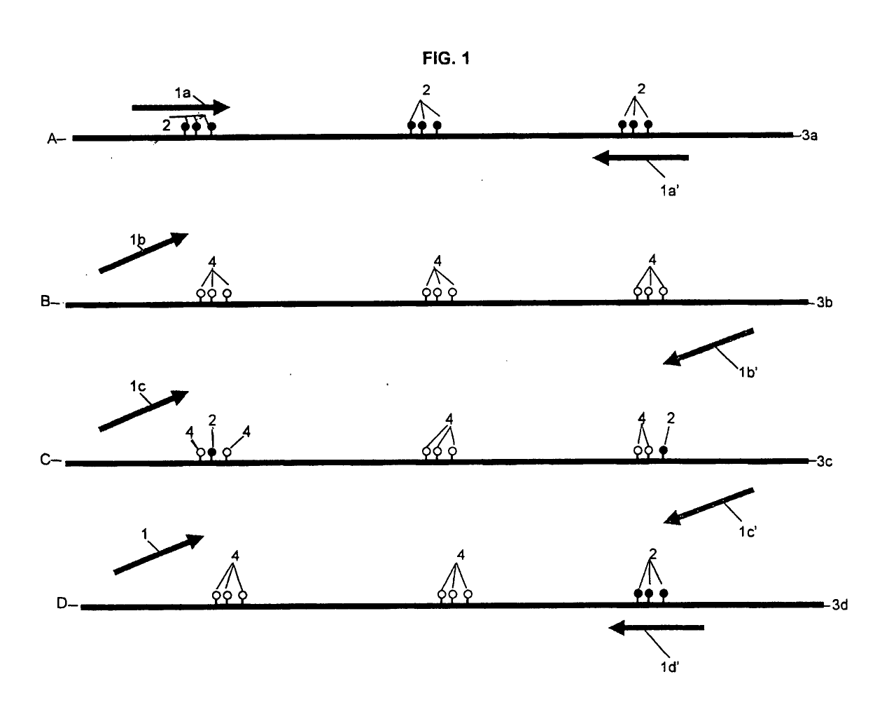

FIG. 1 shows a method of MSP amplification according to the prior art—a typical MSP polymerase mediated amplification of a CpG-rich sequence using methylation specific primers on four representative bisulfite-treated DNA strands (A-D) (“MSP Amplification”). In the method disclosed in Herman et al. (U.S. Pat. No. 5,786,146), primers are complementary to the bisulphite converted target sequence including a CG dinucleotide, i.e., primers hybridize to positions that were methylated in the original nucleic acid sample. Amplificate nucleic acids are then detected, thereby indicating the presence of a methylated nucleic acid in the sample. Alternatively, the primers may comprise a ‘TG’ or ‘CA’ dinucleotide in place of the ‘CG’ dinucleotide, thereby amplifying and enabling the detection of unmethylated versions of the target nucleic acid. In a further illustration of the prior art, the use of the two species of primers may be combined in one reaction suitable for the analysis of heterogeneous samples.

In FIG. 1, methylation specific forward primers 1a, 1b, 1c, and 1d and reverse primers 1a′, 1b′, 1c′, and 1d′ on bisulfite-treated DNA strands A-D, respectively, can anneal to the bisulfite-treated DNA strand 3a if the corresponding subject genomic CpG sequences were methylated. The bisulfite-treated DNA strands can be amplified if both forward and reverse primers anneal, as shown in strand A. Dark circular marker positions (2) on the DNA strands 3a, 3c, and 3d represent methylated bisulfite-converted CpG positions, whereas white circular positions (4) represent unmethylated bisulfite-converted positions.

In the top example, strand A represents the case where all the subject genomic CpG positions were co-methylated, and both forward and reverse primers are thereby able to anneal with and amplify the corresponding treated nucleic acid. For strand B, none of the subject genomic CpG positions were methylated, and therefore none of the primers anneal to the corresponding treated nucleic acid sequence, and the sequence is not amplified. For strand C, the three subject genomic CpG positions covered by the forward and reverse primers are not co-methylated (only one of the positions is methylated), and therefore, subsequent to bisulfite treatment of the DNA the primers do not anneal. For strand D, the positions covered by the reverse primer were methylated CpG sequences in the subject genomic DNA, and the reverse primer thus anneals to the corresponding bisulfite-treated sequence. However, there is no exponential amplification of the corresponding bisulfite-treated DNA sequence, because the subject genomic CpG positions covered by the forward primer were not methylated and the forward primer does not anneal. As disclosed in Laird et al. in U.S. Pat. No. 6,331,393 B1, the method of Herman et al. is not quantitative, since the paired primers used in Herman et al. may represent only one of multiple possible sequence variants and is based on the occurrence or non-occurrence of a PCR product in the fully methylated versus fully unmethylated reaction.

In the prior art, therefore, it can be seen that MSP is neither a sufficiently versatile nor a practical technique for the analysis of complex methylation patterns. For the investigation by MSP of the methylation status of a sequence that may comprise both methylated and non-methylated cytosine positions, a large number of primers covering every possible combination of methylated and non-methylated CpG positions has to be designed and tested. This is economically impractical, time consuming, labor intensive, expensive, and commercially not feasible. These problems would become more apparent were the method to be applied in a high throughput setting, where the maintenance of data quality may be difficult to control and false annealing of primers may lead to incorrect data interpretation. A further drawback of the method as described by Herman et al. is that although the method enables the detection of methylated sequences within a sample, it does not allow a quantification or absolute measurement of the amount of methylated sequences present in a sample.

In contradistinction to the prior art, the current invention provides a method for analysis of methylation patterns within nucleic acids, including analysis of CpG rich islands. In a first embodiment the method provides a means for analysis of the degree of methylation at a single CpG position. This would be applicable where a sample is of heterogeneous origin—for example, where multiple samples from one category are pooled, the described method would provide a means of determining the average occurrence of methylation within the sample set. A second embodiment of the method provides a means for the analysis of multiple CpG positions within a sample such that the relative amounts of methylation at each position is derived by calibration to a reference sequence.

In the first embodiment, methylated cytosine bases in a genomic DNA sample that are unmethylated at the 5-position are converted by treatment with an agent, e.g., bisulfite, to uracil or another base that is dissimilar to cytosine in terms of base pairing behavior. One or more nucleic acids of the treated sample are amplified in an amplification reaction by means of two or more primer oligonucleotide pairs for every CpG position to be analyzed. One primer pair hybridizes preferentially if the CpG position was methylated before bisulfite treatment, and another primer pair hybridizes preferentially if the CpG position was unmethylated before bisulfite treatment. Amplificates formed from each primer pair are detected and measured, allowing determination of the degree of methylation at each analyzed CpG position by comparison of the amounts of the respective amplificates.

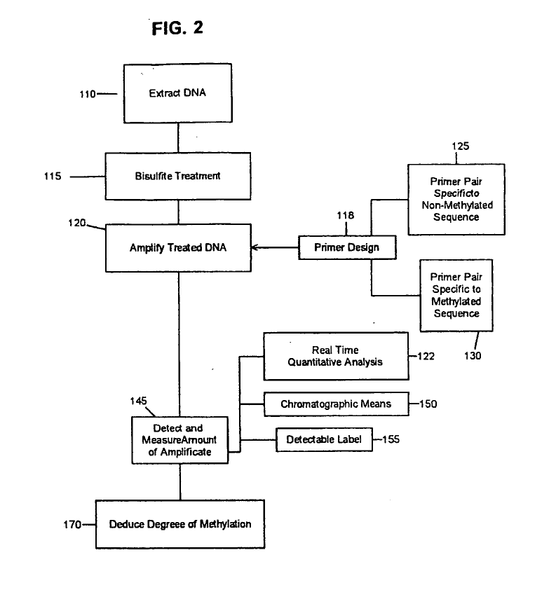

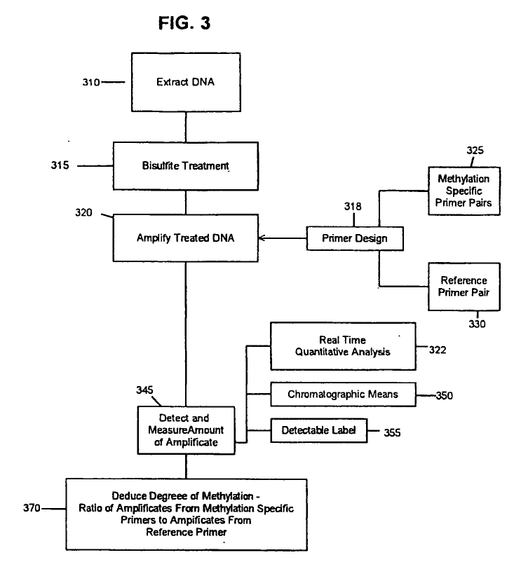

The following description refers, but is not limited, to an embodiment of the invention illustrated by the flow chart in FIG. 2. In accordance with the invention, a sample of nucleic acid, for example DNA, is first extracted from tissue or cellular sources (110), which may include, for example, tissue samples, cell lines, histological slides, body fluids, or tissue embedded in paraffin. The term “nucleic acid” refers to deoxyribonucleotides or ribonucleotides and polymers thereof in either single- or double-stranded form. The term encompasses nucleic acids containing known nucleotide analogs or modified backbone residues or linkages whether synthetic, naturally occurring, or non-naturally occurring, which have similar binding properties as a reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs).

For purposes of illustration, DNA will be referred to in the illustration of the embodiment of the invention in FIG. 2. Extraction may be by means that are standard to one skilled in the art—including, for example, the use of detergent lysates, sonification, and vortexing with glass beads.

The extraction of DNA for further analysis can take place in a minute volume, usually in a layer of oil, which prevents contact with the environment and keeps keep losses of DNA low to provide a reproducible result even with small starting quantities.

In step (115), the genomic DNA sample is treated with a converting agent such that cytosine bases that are unmethylated at the 5′-position are converted to uracil, thymidine, or another base which is dissimilar to cytosine in terms of hybridization behavior, referred to here as “pre-treatment.” Examples of converting agents are sulfite and disulfite solutions. A preferred agent is sodium bisulfite (NaHSO₃), which reacts with the 5,6-double bond of cytosine and proceeds at a significantly slower rate than reaction with methylated cytosine bases. Cytosine reacts with the bisulfite ion to form a sulfonated cytosine reaction intermediate that is susceptible to deamination, giving rise to a sulfonated uracil. The sulfonate group can be removed under alkaline conditions, resulting in the formation of uracil.

Since uracil is recognized as thymine by polymerase, upon polymerase chain reaction (PCR) the resultant product contains cytosine only at the position where 5-methylcytosine occurs in the initial template DNA. The conversion of a genomic sequence by bisulfite treatment commonly results in the creation of a cytosine poor nucleic acid.

In the most preferred embodiment of the method, the bisulphite treatment is carried out according to the agarose matrix as described by Olek A., Oswald, J., and Walter J., “A modified and improved method for bisulphite based cytosine methylation analysis,” Nucleic Acids Res., Dec. 15, 1996 24(24):5064-6. In this method cells or isolated chromosomal DNA are enclosed in an agarose gel matrix prior to bisulphite treatment. By enclosing the nucleic acids to be analysed in a solid matrix loss of sample DNA is limited and sensitivity of the method is thereby increased. Furthermore, it has been observed that the agarose bead method of bisulphite treatment has a higher conversion rate than other methods.

The different reaction steps of the bisulfite reaction are equilibrium reactions, in which the equilibria for the two important reaction steps, the sulfonation of the cytosine and the subsequent deamination, are on the correct (sulfonated and deaminated) side at different temperatures. Therefore, it is advantageous to carry out the bisulfite reaction under cyclic conditions with changing temperatures. A preferred embodiment of the method comprises a change from about 4° C. (10 min) to 50° C. (20 min). All the other temperatures, and reaction times at certain temperatures, however, should be included in the method according to the invention. For example, under certain conditions, it has been advantageous if considerably shorter reaction times are regulated. It is also useful to insert a step at which the DNA to be examined is again denatured at very high temperature, between a deamination step (at high temperature, ≧50° C.) and a subsequent repeated sulfonation step. For high molecular weight DNA, the denaturation temperatures are generally >90° C., but they can also be lower. In some variations of the method, DNA fragments to be examined are very short. In other cases, the complementarity between strands can decrease. In a cyclic reaction protocol, the denaturation temperature in the first cycle can be higher than 90° C., but in later cycles it can be regulated to lower values.

DNA extraction may be carried out, as described in Olek et al., in a minute volume under an oil layer, for example 1 μL but also with smaller or larger volumes. After the DNA sample is denatured, the required bisulfite concentration is then added by the addition of a larger volume of a bisulfite solution (for example, 4 μL), which is slightly larger than necessary for the proper treatment, so that the required final concentrations and pH become automatically established under the oil. Subsequently, the bisulfite reaction is carried out in one of the described manners.

According to an embodiment of the invention, the pretreated nucleic acid is then used as the basis for a CpG site analysis based on an amplification reaction, such as polymerase. An “amplification reaction” refers to any chemical, including enzymatic, reaction that results in increased copies of a template nucleic acid sequence, such as but not limited to PCR. In this context, thermostable polymerases of any origin can be used. The type of the polymerase used is not essential, and it can also be varied depending on the existing buffer conditions. This solution contains such a polymerase and all nucleotides and required oligonucleotide primers. After the addition of this solution, an amplification can thus take place directly in the same reaction vessel.

The amplification may be carried out using two or more oligonucleotide primer pairs, wherein two primer pairs are used in the analysis of each CpG position. In a preferred embodiment of the method, two primer pairs (125, 130) are used in the amplification of each target sequence, wherein the target sequence comprises at least one CpG position to be analysed. One primer pair anneals specifically to target sequence that was unmethylated prior to the bisulphite treatment, and the other primer pair anneals to the same target sequence in the case that the sequence was methylated prior to the bisulphite treatment.

According to one embodiment of the invention, each pair of amplification primers consists of a first (forward) primer and a second (reverse) primer, as in standard in many amplification methods, such as polymerase chain reaction PCR). Each of the primer pairs is required to consist of at least one methylation specific primer oligonucleotide. A methylation specific primer refers to a primer oligonucleotide for use in the amplification of a methylation discriminating bisulfite treated nucleic acid (or similarly converted nucleic acid), wherein the primer contains at least one CpG or CpA dinucleotide within its sequence. As described in, for example, U.S. Pat. No. 5,796,146 to Herman et al., MSP primers consist of an oligonucleotide specific for annealing to a nucleotide sequence containing at least one bisulphite treated CpG dinucleotide. Therefore, according to this embodiment of the method, the primer pair that hybridizes preferentially to the target nucleic acid that was methylated prior to the bisulfite treatment comprises a CpG dinucleotide at the CpG position to be investigated, and the primer pair that hybridises preferentially to the target nucleic acid that was unmethylated prior to the bisulfite treatment comprises a TpG or CpA dinucleotide at the CpG position. Methylation specific primers generally contain relatively few cytosines, as cytosines are converted by the bisulphite reaction. However, when the primers are specific for methylated cytosine dinucleotides, cytosine positions are conserved within the primer oligonucleotides. Therefore, the sequence of the primers includes at least one CpG, TpG, or CpA dinucleotide. MSP primers generally contain relatively few cytosines, as cytosines are converted by the bisulphite reaction. However, when the primers are specific for methylated cytosine dinucleotides, cytosine positions are conserved within the primer oligonucleotides.

The design of methylation specific primers may be carried out using software such as “Primo MSP 3.4.” It is preferred that the design of methylation specific primers be carried out according to the following guidelines:
Primers should contain at least one CpG site within their sequence, and the CpG site should preferably be located in the most 3′-end of their sequence to discriminate methylated DNA against unmethylated DNA.
Primers should have a minimal number of non-CpG cytosines in their sequence to amplify only bisulfite converted DNA. Primers with more non-CpG cytosines are preferred, since the bisulfite conversion may on some occasions be incomplete.
The set of primers for methylated DNA and the set for unmethylated DNA should contain the same CpG sites within their sequence. For example, if a forward primer for methylated DNA has this sequence: ATTAGTTTCGTTTAAGGTTCGA, the forward primer for unmethylated DNA must also contain the two CpG sites as the methylated forward primer. However, they may differ in length and start position.
Both sets of primers should have similar annealing temperature.

In a preferred embodiment, all primers are then extended, preferably by means of a polymerase reaction. Wherein a polymerase is used, the resultant double-stranded nucleic acid is denatured, preferably by means of heat treatment. Successive cycles of primer annealing, extension, and denaturation are carried out according to the polymerase chain reaction, as is known in the art.

In the above embodiments, the amplificates formed from each of the primer pairs are measured and the amount of the amplificates formed from each primer pair is determined.

The amplification reaction (120) produces amplificates from all species of primers, which are distinguishable respectively from those formed from other primer pairs. Therefore, it is required that each species of amplificate is differentiable from other species of amplificates by means of, for example, their length, sequence, or a detectable label (155).

Where the amplificates are separated by length, this may be accomplished by any chromatographic means standard in the art (150), for example gel electrophoresis. The separated fragments may then be visualized e.g. by ethidium bromide staining or by means of hybridisation with labelled probes (Southern Blot), and the amounts of one species of amplificate relative to another may then be determined by the respective size of the bands. In determining the degree of methylation at each analyzed CpG position, the amounts of one species of amplificate relative to another may then be judged by the respective size of the bands.

In a further embodiment according to the invention, the subsequent measurement of the occurrence of each species of amplificate in the solution may be carried out by means of the incorporation of a detectable label (155). This can be achieved by labelling each species of primer. Different forms of labels may be used, but preferred are, fluorescence labels, radionuclides, or detachable molecule fragments having a specific mass which can be detected in a mass spectrometer.

Fluorescent labelled nucleic acids are commonly used in the field and a wide variety of fluorescent molecules are suitable for use in the method according to the invention. The attachment of the fluorescent labels to the primers is within the skill of the art. The simple attachment of Cy3 and Cy5 dyes to the 5′-OH of the primers are particularly suitable for fluorescence labels. Cy3 and Cy5 dyes, are commercially available, as are many suitable fluorescent molecules. The detection (145) of the fluorescence of the hybridized probes may be carried out, for example via a confocal microscope. Alternatively, the amplificate synthesis may be observed in a real time manner by means of fluorescence polarisation as described for example in German Pat. Nos. DE 101 04 938 and DE 100 65 814, Berlin, K., and Distler, J.

A wide variety of fluorophores are suitable for use in fluorescence polarisation techniques. The selection of appropriate fluorophores is within the skill of the art. Preferred fluorophores include, but are not limited to, 5′carboxyfluorescein (FAM) 6-carboxy-X-rhodasine (ROX); N,N,N′,N′,-tetramethyl-6-carboxy-X-rhodamine (TMR); BODIPY-Texas Red (BTR), CY5, CY3 , FITC, DAPI, HEX, and TET. The length of the linkers used to attach the fluorophores to the bases of the nucleic acids should be minimized while achieving maximum rigidity. Short and/or rigid linkers minimize the movement of the fluorophore relative to the oligonucleotide, thus increasing the sensitivity of the assay. The sensitivity of the fluorescence polarization detection may be further increased by decreasing the rotational motility of the bisulfite treated DNA or the primer by increasing their mass. The sensitivity of the fluorescent polarization detection may be further increased by decreasing the rotational motility of the bisulfite treated DNA or the primer by increasing their mass.

In the case of mass labels, visualization may be carried out by means of matrix assisted laser desorption/ionization mass spectrometry (MALDI) or using electron spray mass spectrometry (EST). MALDI-TOF (time of flight) spectrometry is particularly suited to the analysis of biomolecules. In MALDI-TOF spectrometry, the selection of the matrix plays an eminently important role. The sensitivity of this method may be further increased by chemically modifying the amplificates in such a manner that they becomes more similar to peptides. Phosphorothioate nucleic acids in which the usual phosphates of the backbone are substituted with thiophosphates can be converted into a charge-neutral DNA using simple alkylation chemistry (Gut I G, Beck S. A procedure for selective DNA alkylation and detection by mass spectrometry. Nucleic Acids Res. Apr. 25, 1995; 23(8):1367-73). The coupling of a charge tag to the modified DNA results in an increase in sensitivity to the same level as that found for peptides. A further advantage of charge tagging is the increased stability of the analysis against impurities which make the detection of unmodified substrates considerably more difficult.

In a further preferred embodiment, the detection (145) step for synthesized amplificates may be carried out concurrently with the amplification reaction (120). For example, PCR amplification may be carried out such that all amplificates carry a detectable label. A variety of labels standard in the art may be used, including but not limited to, fluorescence labels, in particular fluorescence polarization labels. In this embodiment of the method, the amplificates are detected by means of oligonucleotide probes that are hybridized to the bisulfite treated DNA concurrently with the amplification of the nucleic acid sample.

A particularly preferred embodiment of this method is the use of fluorescence-based Real Time Quantitative PCR (Heid et al., Genome Res. 6:986-994, 1996; see also U.S. Pat. No. 6,331,393 B1, Laird et al.). There are two preferred embodiments of utilising this method. One embodiment, known as the TaqMan™ assay employs a dual-labelled fluorescent oligonucleotide probe. The TaqMan™ PCR reaction employs the use of a nonextendible interrogating oligonucleotide, called a TaqMan™ probe, which is designed to hybridise to a target sequence located between the forward and reverse amplification primers. The TaqMan™ probe further comprises a fluorescent “reporter moiety” and a “quencher moiety” covalently bound to linker moieties (e.g., phosphoramidites) attached to the nucleotides of the TaqMan™ oligonucleotide. Hybridized probes are displaced and broken down by the polymerase of the amplification reaction thereby leading to an increase in fluorescence. For analysis of methylation within nucleic acids subsequent to bisulfite treatment, it is further preferred that the probe be methylation specific, as described in Laird et al. (hereby incorporated by reference in its entirety), also known as the MethyLight™ assay. The second preferred embodiment of this technology is the use of dual-probe technology (Lightcycler™). Each probe carries a donor or recipient fluorescent moieties, hybridization of two probes in proximity to each other is indicated by an increase or fluorescent amplification primers. This technique may also be adapted in a manner suitable for methylation analysis of CpG dinucleotides within the amplificates.

The degree of methylation is deduced (170) at each CpG position from the ratio of the amount of amplificate formed from methylated and non-methylated primers at each analyzed CpG position.

In the second preferred embodiment, illustrated in FIG. 3, at least three primer oligonucleotide pairs are used in the amplification. Extraction (310), bisulfite treatment (315), and amplification (320) are carried out as in the first embodiment described with reference to FIG. 2. One primer pair (330) amplifies a reference sequence and at least two primer pairs are specific to a target CpG positions or positions (325). Amplificates formed from the primer pairs are detected and measured (345) as described for the embodiment illustrated in FIG. 2 (145). Real time quantitative analysis (322) is utilized for detection and measurement of amplificates (345). In step (370), the degree of methylation within the analyzed sequence is deduced from the ratio of the amount of amplificate formed from each methylation specific primers to the amount of amplificate formed from the reference primer.

In a third preferred embodiment, the invention provides a method for the analysis of the methylation status of one or more CpG dinucleotides within a nucleic acid sample. The nucleic acid sample to be analyzed is treated with a converting agent such that cytosine bases that are unmethylated at the 5-position are converted to uracil or another base which is dissimilar to cytosine in terms of base pairing behavior. Methylation specific primer pairs are used amplifying one or more target nucleic acids of the treated nucleic acid and one or more reference samples. The amplification is carried out by means of a polymerase enzyme reaction by means of one or two methylation specific primer oligonucleotide pairs per CpG position such that the amplificates formed from each species of primer pair differ respectively in at least one of length, sequence, and a detectable label selected from a group consisting of fluorescence labels, mass labels, and radioactive labels. The amplificates formed from the primer pairs within each sample are detected, and the amounts of the amplificates formed from each primer pair in each of the samples are measured. The method the amount of methylation within the treated sequence at each CpG position that was analyzed is deduced by determining the amount of amplificate formed within the treated sample relative to the amount of amplificate formed within the reference samples(s) for each primer pair.

In this embodiment, the reference samples may be either a full methylated version of the sequence to be analyzed or a fully unmethylated version of the sequence to be analyzed. However, it is particularly preferred that both reference samples are used. The fully methylated reference sample may be prepared by any available means or supplied from a commercial source. In one embodiment it may be prepared by treating a sample of nucleic acid (from clinical sources or commercial suppliers) with the enzyme SssI Methylase and a suitable methyl donor co-factor such as S-adenosylmethionine in an appropriate buffer solution (for example Mssl-Buffer) at an appropriate temperature (preferably 37 degrees) for an appropriate length of time (for example but not limited to sixteen hours). The fully unmethylated reference sample may be prepared by any available means or supplied from a commercial source. In one embodiment it may be prepared by enzymatic amplification of a sample nucleic acid (from clinical sources or commercial suppliers) using standard means such as the polymerase chain reaction.

Bisulfite treatment of test and reference samples are carried out as in previously described embodiments. The treated test sample and the reference sample(s) are then amplified using one or more primer pairs. It is preferred that each CpG position is analyzed using both the methylated strand specific primer pairs (i.e., wherein the primer comprises one or more CpG dinucleotides), and the unmethylated strand specific primer pair (i.e., wherein the primer comprises one or more TpG or CpA dinucleotides). However, the method is still enabled by the use of only one species of primer.

Design of primers for the amplification is carried out as in previous described embodiments, as are amplification conditions. Amplificates formed from each of the primer pairs in each of the samples are detected, and the amount of the amplificates is determined by methods for the detection of amplificates as in previous embodiments. The amounts of amplificate synthesized from each of the samples are then compared relative to each other for purposes of quantification.

The comparison of amplificates is carried out analogous to the second embodiment described above. In a particularly preferred embodiment the amplification is monitored by means of a real time manner. A first calibration curve for each species of primer is plotted wherein the amount of amplificate within each sample (both reference and treated) is plotted against cycle number. From this plot, the crossing line is determined, the crossing line being the point on each curve at which the PCR cycle amplification signal enters the log linear phase. Using these intersection points, a calibration graph can be calculated which defines a relationship between the cycle number at which the amplification signal intersects the crossing line and the template concentration initially present in the sample. Thus, if the intersection point of an amplification signal (expressed as the cycle number) is known, the initial template concentration can be directly derived from the calibration graph. These calculations may be calculated using the Fit Points and Second Derivative Maximum Methods. The Second Derivative Maximum Method is preferred if samples with a high copy number (above 1000 copies/sample) are to be analyzed. If samples with a low copy number are to be analyzed, the Fit Points Method is preferred.

Where a reference primer has been employed, amplificates from all other primer pairs are normalized by comparison to the amplificate that is synthesized from the reference primer, thus allowing for a relative comparison of the amount of methylated as opposed to non-methylated target DNA present in the sample.

In a further embodiment of the method, the amount of amplificate from each MSP primer pair is compared to the amount of amplificate synthesized from the other MSP primers and to the amount of amplificate synthesized from the reference primer. Amplificates from all other primer pairs are normalized by comparison to the amplificate that is synthesized from the reference primer. The amounts of amplificate synthesized from each of these primer pairs are then compared relative to each other, thus allowing for a relative quantification of the degree of methylation within a selected region of the genome (370).

In a further embodiment of the method, the amount of amplificate from each MSP primer pair is compared to the amount of amplificate synthesized from the other MSP primers and to the amount of amplificate synthesized from the reference primer. By comparison to the amount of amplificate formed from the reference primer, it is possible to normalize the levels of methylation at a particular position to the level of non-methylation. The method thereby allows one to both quantitatively assess the degree of methylation at a specific position and establish the relative levels of methylation at specific positions within a selected region of the genome.

Embodiments of the invention described above may be adapted according to various uses. In one embodiment, the method may be used to analyze the degree of methylation at a specific CpG dinucleotide position. Such use allows for analysis of heterogeneous nucleic acid samples—i.e., samples containing both nucleic acid molecules that are methylated and nucleic acid molecules that are unmethylated at the position in question. Clinical samples of solid tumors, such as breast tumors, often consist of multiple tissue types (i.e., tumor and surrounding tissues). The amplification reaction is carried out using two primer pairs per CpG dinucleotide position (as per a standard MSP reaction). One primer pair is specific for the methylated version of the CG dinucleotide and therefore contains a CG at the position in question, and the other primer pair is specific for the unmethylated version of the CG position in question and therefore contains a TG dinucleotide at the position in question. Amplification of a nucleic acid sample using the methylation and non-methylation specific primers results in the formation of a mixed population of amplificates wherein the relative levels of methylation at the CpG positions in question are assessed by comparison to the levels of amplificate synthesized from the reference primer oligonucleotide.

In an alternative embodiment the described method may be applied to the investigation of large populations, for example in research investigations of pooled samples. By pooling samples and investigating them using the described method it is possible to deduce in a time and cost effective manner the incidence of methylation at the investigated CpG positions in a single reaction. Another embodiment of the invention includes a kit useful for performing the polynucleotide amplification reaction described herein. Such a kit may include (1) a converting reagent, preferably a solution of sodium disulfite or hydrogen sulfite, (2) at least two pairs of oligonucleotide primers for the amplification of bisulfite treated nucleic acids, (3) reagents for carrying out the polynucleotide amplification reaction, the reagents including deoxynucleotide triphosphates and a DNA polymerizing enzyme, and (4) instructions for carrying out the amplification reaction and for specifically detecting the amplificates. Optionally, the kit may further comprise detectably labeled oligonucleotide probe molecules for use in quantification of methylation within the samples.

The following examples further illustrate aspects of the invention.

### EXAMPLE 1

The following example describes the analysis of a CpG rich island within the 5′region of the gene TPEF (NM 016192). Hypermethylation of this region has previously been associated with tumorigenesis in tumor cell lines (Cancer Research 60, 4907-4912, Sep. 1, 2000).

DNA may be extracted using a suitable commercially available kit e.g. Qiagen™ extraction kit. The DNA sample is then treated using a bisulfite solution (hydrogen sulfite, disulfite) according to the agarose-bead method (Olek et al 1996). The treatment is such that all non methylated cytosines within the sample are converted to thymidine. Conversely, 5-methylated cytosines within the sample remain unmodified. The methylation status is determined with a methylation specific assay designed for the CpG island of interest. The CpG island assay covers CpG sites in both the primers and the Taqman™ style probe. Methods:

The assay specific to the methylated version of the CpG island is performed using the following primers and probes:
Primer: TTTTCGTCGTTTAGGTTATCG (SEQ ID NO:1); Primer: TTTTTGTTGTTTTAGGTTATTGG (SEQ ID NO:2); and Probe: TTCGGACGTCGTTGTTCGGTCGATGT (SEQ ID NO:3). The corresponding assay specific to the unmethylated version of the CpG island is performed using the following primers and probes: Primer: TTTTTGTTGTTTTAGGTTATTGG (SEQ ID NO:4); Primer: CATATGCTGTGAATAAATTAC (SEQ ID NO:5); and Probe: TTTGGATGTTGTTGTTTGGTTGATGT (SEQ ID NO:6) The reaction is run with the following assay conditions: Reaction solution: (900 nM primers; 300 nM probe; 3.5 mM Magnesium Chloride; 1 unit of taq polymerase; 200 μM dNTPs; 7 μl of DNA, in a final reaction volume of 20 μl); Cycling conditions: (95° C. for 10 minutes; then 50 cycles of: 95° C. for 15 seconds; 60° C. for 1 minute). The reaction is observed in real time by use of commercially available instruments such as the ABI PRISM 7700 sequence detector.

The relative amounts of amplificate formed are then used to deduce the relative levels of methylated as opposed to non methylated nucleic acids in the sample.

### EXAMPLE 2

The following example describes the analysis of a CpG rich island within the 5′region of the gene Calcitonin. Investigation of the Calcitonin gene has revealed that hypermethylation of the promoter region of the gene is present in neoplastic cells of several cancer types, particularly acute leukemias.

DNA may be extracted using a suitable commercially available kit, e.g. Qiagen™ extraction kit. The DNA sample is then treated using a bisulfite solution (hydrogen sulfite, disulfite) according to the agarose-bead method (Olek et al 1996). The treatment is such that all non methylated cytosines within the sample are converted to thymidine. Conversely, 5-methylated cytosines within the sample remain unmodified. The methylation status may be determined with a methylation specific assay designed for the CpG island of interest and a reference fragment.

The assay specific to the methylated version of the CpG island is performed using the following primers and probes:
Primer: CGGATACGATTTCGGGG (SEQ ID NO:7); Primer: ATACGATAAACGCAACAACGAC (SEQ ID NO:8); and Probe: ATTTGGAGTTTCGTGATTCGCGTTACGGA (SEQ ID NO:9). The corresponding assay specific to the unmethylated version of the CpG island is performed using the following primers and probes: Primer: TGGATATGATTTTGGGGTA (SEQ ID NO:10); Primer: ATATGATAAATGCAACAATGACAT (SEQ ID NO:11); and Probe: ATTTGGAGTTTTGTGATTTGTGTTATGGA (SEQ ID NO:12)

The corresponding reference assay was performed using the following primers and probes: Primer: TCCATATTCCAAACCCTATACCAAA (SEQ ID NO:13); Primer: TGGGATTGAGGGTAAGAGGGAT (SEQ ID NO:14). The reaction is run with the following assay conditions: Reaction solution: (900 nM primers; 300 nM probe; 3.5 mM Magnesium Chloride; 1 unit of taq polymerase; 200 μM dNTPs; 7 μl of DNA, in a final reaction volume of 20 μl); Cycling conditions: (95° C. for 10 minutes; then 50 cycles of: 95° C. for 15 seconds; 60° C. for 1 minute ). The reaction is observed in real time by use of commercially available instruments such as the ABI PRISM 7700 sequence detector.

The amount of methylated nucleic acid in the tumor sample is quantified by plotting a first calibration curve wherein the amount of amplificate from each sample is plotted against cycle number. From this plot, the crossing line is determined, the crossing line being the point on each curve at which the PCR cycle amplification signal enters the log linear phase. Using these intersection points a calibration graph can be calculated which defines a relationship between the cycle number at which the amplification signal intersects the crossing line and the template concentration initially present in the sample. Thus, if the intersection point of an amplification signal (expressed as the cycle number) is known, the initial template concentration can be directly derived from the calibration graph. These calculations may be calculated using the Fit Points and Second Derivative Maximum Methods. The Second Derivative Maximum Method is preferred if samples with a high copy number (above 1000 copies/sample) are to be analyzed. If samples with a low copy number are to be analyzed, the Fit Points Method is preferred.

### EXAMPLE 3

In the following example, the methylation specific assay according to Example 2 is run on three different samples.

A first sample is obtained from a tumor sample, and is isolated using a suitable commercially available kit e.g. Qiagen™ extraction kit.

A second genomic DNA sample commercially available from Promega is artificially methylated using the following method:

Reagents:

DNA
SssI Methylase (concentration 2 units/μl).
SAM (S-adenosylmethionine)
4.5 μl Mssl-Buffer (NEB Buffer B+ (10 mM Tris-HCl 300 mM NaCl, 10 mM Tris-HCl, 0.1 mM EDTA, 1 mM dithiothreitol, 500 μg/ml BSA, 50% glycerol (pH 7.4 at 25° C.) pH 7.5; 10 mM MgCl₂; 0.1 mg/ml BSA)
dd water (0.2 μm-filtered autoclaved, DNases, RNases, proteases, phosphatases-free).
   Method:

Reagents are combined and incubated at 37 degrees for 16 hours. The sample may then be stored in the refrigerator (+4° C.).

A third genomic DNA sample commercially available from Promega is amplified by means of a polymerase reaction using the following reagents to ensure that no methylation is present in the sample.

All three samples are then bisulphate treated according to the agarose bead method.

The assay specific to the methylated version of the CpG island is performed using the following primers and probes:
Primer: CGGATACGATTTCGGGG (SEQ ID NO:7); Primer: ATACGATAAACGCAACAACGAC (SEQ ID NO:8); and Probe: ATTTGGAGTTTCGTGATTCGCGTTACGGA (SEQ ID NO:9). The corresponding assay specific to the unmethylated version of the CpG island is performed using the following primers and probes: Primer: TGGATATGATTTTGGGGTA (SEQ ID NO:10); Primer: ATATGATAAATGCAACAATGACAT (SEQ ID NO:11); and Probe: ATTTGGAGTTTTGTGATTTGTGTTATGGA (SEQ ID NO: 12)

Each reaction is run with the following assay conditions: Reaction solution: (900 nM primers; 300 nM probe; 3.5 mM Magnesium Chloride; 1 unit of taq polymerase; 200 μM dNTPs; 7 μl of DNA, in a final reaction volume of 20 μl); Cycling conditions: (95° C. for 10 minutes; then 50 cycles of: 95° C. for 15 seconds; 60° C. for 1 minute ). The reaction is observed in real time by use of commercially available instruments such as the ABI PRISM 7700 sequence detector. The amount of methylated nucleic acid in the tumor sample is quantified by plotting a first calibration curve wherein the amount of amplificate from each sample is plotted against cycle number. From this plot, the crossing line is determined, the crossing line being the point on each curve at which the PCR cycle amplification signal enters the log linear phase. Using these intersection points a calibration graph can be calculated which defines a relationship between the cycle number at which the amplification signal intersects the crossing line and the template concentration initially present in the sample. Thus, if the intersection point of an amplification signal (expressed as the cycle number) is known, the initial template concentration can be directly derived from the calibration graph. These calculations may be calculated using the Fit Points and Second Derivative Maximum Methods. The Second Derivative Maximum Method is preferred if samples with a high copy number (above 1000 copies/sample) are to be analyzed. If samples with a low copy number are to be analyzed, the Fit Points Method is preferred.

Other aspects of the invention will become apparent upon review of the following description of preferred embodiments of the invention, taken in conjunction with the accompanying drawings. The invention, however, is pointed out by the appended claims.

1. A method for analyzing methylation at one or more CpG positions to be analyzed in a nucleic acid sample, comprising:
a. converting unmethylated cytosine bases in the nucleic acid sample by treatment with an agent to uracil or another base that is dissimilar to cytosine in terms of base pairing behavior;
b. amplifying one or more nucleic acids of the treated sample in an amplification reaction, wherein at least two oligonucleotide primer pairs are provided for every CpG position to be analyzed, one of which primer pairs hybridizes preferentially in the case where the CpG to treated nucleic acid was methylated in the original sample before conversion, and further wherein the other of which primer pairs hybridizes preferentially in the case where the CpG to treated nucleic acid was unmethylated in the original sample before conversion;
c. detecting the amplificates formed in the polymerase reaction in a quantifiable manner; and
d. determining the degree of methylation in at least one CpG position of the nucleic acid sample.

2. The method of claim 1, wherein the converting agent is bisulfite or a compound thereof.

3. The method of claim 1, wherein amplification is carried out by a polymerase enzyme.

4. The method of claim 1, wherein the amplificates formed from each primer pair differ from those formed by another primer pair in at least one of length, sequence, and a detectable label.

5. The method of claim 4, wherein the detectable label is selected from a group consisting of fluorescence labels, mass labels, and radioactive labels.

6. The method of claim 1, wherein detecting the amplificates is carried out by means of mass spectrometry.

7. The method of claim 1, wherein detecting the amplificates is carried out by means of a real time technique.

8. A method for the analysis of the methylation status of one or more CpG dinucleotides within a nucleic acid sample, comprising:
a. in the nucleic acid sample, converting cytosine bases that are unmethylated at the 5-position by treatment with a converting agent to uracil or another base which is dissimilar to cytosine in terms of base pairing behavior;
b. amplifying one or more nucleic acids of the treated nucleic acid in a polymerase enzyme reaction by means of at least two primer oligonucleotide pairs, wherein one primer pair amplifies a reference sequence and the other primer pairs are methylation specific primers, and further wherein the amplificates formed from each species of primer pairs differ respectively in at least one of length, sequence, and a detectable label selected from a group consisting of fluorescence labels, mass labels, and radioactive labels;
c. detecting the amplificates formed from the primer pairs;
d. measuring the amounts of the amplificates formed from each primer pair; and
e. determining the degree of methylation at each analyzed CpG position.

9. The method of claim 8, wherein the converting agent is bisulfite or a compound thereof.

10. The method of claim 8, wherein the detection of amplificates is carried out by one of mass spectrometry and a real time technique.

11. The method of claim 10, wherein detection by mass spectrometry is carried out by means of matrix assisted laser desorption/ionization mass spectrometry (MALDI) or electron spray mass spectrometry (EST).

12. The method of claim 10, wherein detection by real time is carried out by means of one or more methods taken from the group comprising Real-Time PCR™ assay, TaqMan™ assay, LightCycler™ assay.

13. The method of claim 8, wherein at least three pairs of primers are used in the polymerase reaction, one of which primer pairs is a reference primer pair that amplifies a non-methylated sequence that acts as a reference sequence.

14. The method of claim 13, wherein the reference primer does not contain a CpG dinucleotide and does not contain a TpG dinucleotide.

15. The method of claim 13, wherein the primer pairs that do not amplify the reference sequence include one or more of CpG, TpG, and CpA dinucleotides.

16. The method of claim 13, wherein the amplificate synthesized from each primer pair is compared to the amplificate from the other primers and to the amount of amplificate from the reference primer.

17. The method according to claim 13, wherein determining the degree of methylation is carried out by determining the amount of each amplificate from each primer pair relative to the amount of amplificate formed from the reference primer pair.

18. The method of claim 13, wherein the amplificates are modified in such a manner that they become similar to peptides.

19. A method for the analysis of the methylation status of one or more CpG dinucleotides within a nucleic acid sample, comprising:
a. converting cytosine bases that are unmethylated at the 5-position by treatment with a converting agent to uracil or another base that is dissimilar to cytosine in terms of base pairing behavior;
b. amplifying one or more nucleic acids of the treated nucleic acid and of one or two reference samples in a polymerase enzyme reaction by means of one or more methylation specific primer oligonucleotide pairs, wherein the amplificates formed from each species of primer pair differ respectively in at least one of length, sequence, and a detectable label selected from a group consisting of fluorescence labels, mass labels, and radioactive labels;
c. detecting the amplificates formed from the primer pairs within each sample
d. measuring the amounts of the amplificates formed from each primer pair in each of the samples; and
e. determining the amount of methylation within the treated sequence by determining the amount of amplificate formed within the treated sample relative to the amount of amplificate formed within the reference sample or samples for each primer pair.

20. The method of claim 19, wherein the converting agent is bisulfite or a compound thereof.

21. The method of claim 20, wherein the reference sample or samples is one of a fully methylated version of the target nucleic acid to be analyzed and a filly unmethylated version of the target nucleic acid to be analyzed.

22. The method of claim 20 wherein the detectable labels are selected from the group consisting of fluorescence labels, mass label, radioactive labels.

23. The method of claim 20, wherein detecting the amplificates is carried out by means of matrix assisted laser desorption/ionization mass spectrometry (MALDI) or using electron spray mass spectrometry (ESI).

24. The method of claim 20, wherein detecting the amplificates is carried out by means of one or more methods taken from the group comprising Real-Time PCR™ assay, TaqMan™ assay, LightCycler™ assay.

25. A plurality of oligonucleotide primer pairs for the determination of the degree of methylation at one or more CpG positions in a nucleic acid sample, wherein the oligonucleotide primer pairs are capable of distinguishing between methylated and non-methylated nucleic acid in the sample after modification by bisulfite treatment, and further wherein a first primer pair hybridizes preferentially to a modified nucleic acid that was methylated in the nucleic acid sample in the sequence the primer is hybridizing to, and a second primer pair binds preferentially to modified nucleic acid that was methylated in the nucleic acid sample in the sequence the primer is hybridizing to.

26. The primer pairs of claim 25, including at least one reference primer pair that is methylation insensitive.

27. The primer pairs of claim 25, wherein the amplificates synthesized from all species of primers are comparable to each other and differ according to at least one of length, sequence and detectable label and are thereby differentially detectable and quantifiable.

28. The primers of claim 25, wherein the amount of amplificate from each primer pair is compared to the amount of amplificate synthesized from the reference primer pair.

29. A kit providing for analysis of the methylation status of one or more CpG dinucleotides within a nucleic acid sample, comprising apparatus including a plurality of segments, including at least a first segment that contains an agent for converting unmethylated cytosines to another nucleotide base within the nucleic acid sample, a second segment that contains at least two oligonucleotide primer pairs that hybridize with a target polynucleotide sequence and amplify CpG containing nucleic acid, one of which primer pairs hybridizes preferentially to converted nucleic acid that was methylated in the original sample in the sequence the primer is hybridizing to, and a second of which pairs hybridizes preferentially to converted nucleic acid that was unmethylated in the original sample in the sequence the primer is hybridizing to, and instructions for carrying out the conversion and amplification, for detecting the amplificates formed in the polymerase reaction in a quantifiable manner and for determining the degree of methylation in at least one selected segment of the nucleic acid sample.

30. The kit of claim 29, further comprising at least one primer pair that either 1) amplifies a non-methylated sequence that acts as a reference sequence, or 2) is methylation insensitive.


SEQUENCE LISTING (15 sequences)

SEQ ID NO: 1 - DNA, 22 bp, Artificial
  oligonucleotide primer
ttttcgtcgttttaggttatcg

SEQ ID NO: 2 - DNA, 23 bp, Artificial
  oligonucleotide primer
tttttgttgttttaggttattgg

SEQ ID NO: 3 - DNA, 26 bp, Artificial
  oligonucleotide probe
ttcggacgtcgttgttcggtcgatgt

SEQ ID NO: 4 - DNA, 23 bp, Artificial
  oligonucleotide primer
tttttgttgttttaggttattgg

SEQ ID NO: 5 - DNA, 21 bp, Artificial
  oligonucleotide primer
catatgctgtgaataaattac

SEQ ID NO: 6 - DNA, 26 bp, Artificial
  oligonucleotide probe
tttggatgttgttgtttggttgatgt

SEQ ID NO: 7 - DNA, 17 bp, Artificial
  oligonucleotide primer
cggatacgatttcgggg

SEQ ID NO: 8 - DNA, 22 bp, Artificial
  oligonucleotide primer
atacgataaacgcaacaacgac

SEQ ID NO: 9 - DNA, 29 bp, Artificial
  oligonucleotide probe
atttggagtttcgtgattcgcgttacgga

SEQ ID NO: 10 - DNA, 19 bp, Artificial
  oligonucleotide primer
tggatatgattttggggta

SEQ ID NO: 11 - DNA, 24 bp, Artificial
  oligonucleotide primer
atatgataaatgcaacaatgacat

SEQ ID NO: 12 - DNA, 29 bp, Artificial
  oligonucleotide probe
atttggagttttgtgatttgtgttatgga

SEQ ID NO: 13 - DNA, 25 bp, Artificial
  oligonucleotide primer
tccatattccaaaccctataccaaa

SEQ ID NO: 14 - DNA, 22 bp, Artificial
  oligonucleotide primer
tgggattgagggtaagagggat

SEQ ID NO: 15 - DNA, 22 bp, Artificial
  hypothetical sequence
attagtttcgtttaaggttcga